(19) United States
(12) Patent Application Publication
(10) Pub. No.: US 2002/0146777 A1
(43) Pub. Date: Oct. 10, 2002

(54) Canine parathyroid hormone 1 receptor

(76) Inventors: Tessa A. Castleberry, Groton, CT (US); Bihong Lu, Killingworth, CT (US); Thomas A. Owen, East Lyme, CT (US); Steven L. Smock, Baltic, CT (US)

Correspondence Address: Gregg C. Benson, Pfizer Inc., Patent Department, MS 4159, Eastern Point Road, Groton, CT, 06340, US

(21) Appl. No.: 09/943,446

(22) Filed: Aug. 30, 2001

Related U.S. Application Data
(60) Provisional application No. 60/229,170, filed on Aug. 30, 2000.

(51) Int. Cl. C07K 14/635; C07H 21/04; C12N 1/18; C12P 21/02; C12N 5/06

(52) U.S. Cl. 435/69.4; 435/254.2; 435/325; 435/320.1; 530/399; 536/23.5

(57) ABSTRACT

The present invention provides isolated canine PTH1 protein, DNA molecules encoding said protein, vectors and cells carrying said DNA molecules, specific bind partners for the protein, and methods of discovering ligands for canine PTH1.

### CROSS REFERENCE TO RELATED APPLICATION

This application claims the benefit of U.S. Provisional Application No. 60/229,170 filed Aug. 30, 2000.

### FIELD OF THE INVENTION

The present invention relates to canine parathyroid hormone 1 receptor (canine PTH1 or caPTH1 herein), corresponding to that found in canine tissues. In other aspects, the invention covers, inter alia, structural variants of caPTH1, polynucleotides that encode caPTH1 and its structural variants, expression vectors containing the polynucleotides, and cells transformed by the expression vectors.

### BACKGROUND OF THE INVENTION

Parathyroid hormone (PTH) is a major mediator of calcium and phosphate metabolism through its interactions with receptors in kidney and bone. PTH binds with high affinity to two known cell surface receptors (PTH1 and PTH2) that are members of the superfamily of G-protein coupled receptors. Cloning of the cDNAs encoding PTH1 from human, rat, mouse, pig, and opossum has revealed a highly conserved molecule with 7 potential transmembrane domains that is functionally coupled to adenylate cyclase, resulting in elevated intracellular cAMP levels upon activation. We have cloned the canine PTH1 receptor and find that its sequence is very similar to those reported for the other PTH1 cDNAs and that its activation by ligand also results in elevated cAMP levels.

Parathyroid hormone (PTH) is an 84-amino-acid peptide secreted by the parathyroid glands that play an important role in calcium and phosphorous homeostasis. PTH-related peptide (PTHrP), though a product of a distinct gene, shares 8 of 13 amino-terminal residues with PTH and was first identified as the product released from tumors that cause the hypercalcemia of malignancy syndrome. PTH and PTHrP both activate a cloned G-protein coupled plasma membrane receptor (PTH1) in bone and kidney, subsequently activating adenylate cyclase and phospholipase C (Jüppner, 1991, Schipani, 1993). Asecond PTH recognizing receptor (PTH2) has been cloned but does not bind PTHrP (Usdin, 1995, Usdin, 1996) with high affinity. PTH has been demonstrated to be a potent bone anabolic agent in several mammalian species (Dempster, 1993; Dempster, 1995). To further the understanding of the molecular and biochemical actions of PTH in canine bone, as well as to expand our understanding of the cAMP linked PTH1 receptor, we have cloned, expressed, and characterized the canine PTH1 receptor. This receptor is similar to PTH1 receptors from human, mouse, and rat on the molecular level and has functional characteristics in common with these receptors with respect to ligand binding and activation.

Regulation of calcium concentration is necessary for the normal function of the gastrointestinal, skeletal, neurologic, neuromuscular, and cardiovascular systems. PTH synthesis and release are controlled principally by the serum calcium level: a low level stimulates and a high level suppresses both the hormone synthesis and release. PTH, in turn, maintains the serum calcium level by directly or indirectly promoting calcium entry into the blood at three sites of calcium exchange: gut, bone and kidney. PTH contributes to net gastrointestinal absorption of calcium by favoring the renal synthesis of the active form of vitamin D. PTH promotes calcium reabsorption from bone by inhibiting osteoblasts and, indirectly, by stimulating differentiation of the bone-reabsorbing cells, osteoclasts. It also mediates at least three main effects on the kidney: stimulation of tubular calcium reabsorption, enhancement of phosphate clearance, and promotion of an increase in the enzyme that completes synthesis of the active form of vitamin D. PTH exerts these effects primarily through receptor-mediated activation of adenylate cyclase, although receptor-mediated activation of phospholipase C by PTH has also been reported (Hruska et al., J. Clin. Invest. 79:230, 1987).

Disruption of calcium homeostasis may produce many clinical disorders (e.g., severe bone disease, anemia, renal impairment, ulcers, myopathy, and neuropathy) and usually results from conditions which produce an alteration in the level of parathyroid hormone. Hypercalcemia is a condition which is characterized by an elevation in the serum calcium level. It is often associated with primary hyperparathyroidism in which an excess of PTH production occurs as a result of a lesion (e.g., adenoma, hyperplasia or carcinoma) of the parathyroid glands. Another type of hypercalcemia, humoral hypercalcemia of malignancy (HHM), is the most common paraneoplastic syndrome. It appears to result in most instances from the production by tumors (e.g., squamous, renal, ovarian or bladder carcinomas) of a novel class of protein hormone which shares amino acid homology with PTH. These PTH-related proteins (PTHrP) appear to mimic certain of the renal and skeletal actions of PTH and are believed to interact with the PTH receptor in these tissues. PTHrP is normally found at low levels in many tissues, including keratinocytes, brain, pituitary, parathyroid, adrenal cortex, medulla, fetal liver, osteoblast-like cells and lactating mammary tissues. In many HHM malignancies, PTHrP is found in the circulatory system at high levels, thereby producing the elevated calcium levels associated with HHM.

### SUMMARY OF THE INVENTION

To further the understanding of the molecular and biochemical actions of parathyroid hormone and related compounds in bone, kidney, and other tissues, as well as to expand the understanding of the cAMP linked parathyroid hormone receptors, we have cloned, expressed, and characterized the canine PTH1 receptor. The canine PTH1 receptor has functional characteristics in common with the previously reported PTH1 receptors from human and other mammals.

Until the work by the present inventors, no work had been reported on isolating a factor having caPTH1 activity from dogs. Several potential difficulties had to be surmounted before the present inventors succeeded in achieving the present invention. First, although caPTH1 proteins had been isolated from other species, as mentioned above, it was unclear to what extent there would be homology between these known caPTH1 materials and that from dogs. Accordingly, it was unclear whether probes based on caPTH1 proteins associated with other species would be useful in isolating an analogous material from dogs. Second it was unclear whether canine cells would produce sufficient amounts of messenger RNA (mRNA) for a canine PTH1.

In spite of the above uncertainties and other difficulties, the present inventors succeeded in isolating a full cDNA sequence corresponding to a protein having caPTH1 activity in dogs. This discovery established the basis of the present invention.

To further study PTH1 receptor subtype function, a cDNA library was constructed from canine kidney mRNA and screened using the human PTH1 cDNA. Two clones were isolated and sequenced. The longest clone was 2177 bp and contained a single open reading frame of 1785 bp, potentially encoding a protein of 595 amino acids with a predicted molecular weight of 66.4 kD. Sequence analysis of this open reading frame reveals >91% identity to the human PTH1 CDNA at the nucleotide level and >95% identity when the putative canine and human protein sequences are compared. Northern blot analysis revealed high levels of PTH1 in the kidney, with detectable levels in the aorta, heart, and lung. Competition binding studies following transfection of PTH1 into CHO cells using ¹²⁵I-hPTH (1-34) as ligand demonstrated specific displacement by canine PTH (1-34), canine/human parathyroid hormone related peptide (PTHrP) (1-34), and human PTH (1-34). These ligands also resulted in increased levels of cAMP in PTH1 transfected cells but not in parental CHO cells. In contrast, aldosterone secretion inhibiting factor (1-35) did not bind to this receptor and did not result in elevated cAMP levels. Together, these data indicate that we have cloned the canine PTH1 receptor and that it is very similar, both in sequence and in functional characteristics, to the other known PTH1 receptors.

Thus, a canine cDNA has been isolated that encodes an open reading frame with a DNA and predicted protein sequence that is highly homologous to the reported PTH1 receptor from human and other species. When this canine cDNA is expressed in CHO cells, it encodes a protein with ligand binding properties characteristic of PTH1 and, when activated by appropriate ligands, results in the accumulation of cAMP within cells. The cloning of canine PTH1 will aid in furthering the understanding of the molecular and biochemical actions of PTH and PTHrP in canine bone.

The present invention has several aspects. In a first aspect, the present invention relates to isolated proteinaceous molecules having an activity of caPTH1 and comprising an amino acid sequence corresponding to SEQ ID NO:2.

In another aspect, the present invention relates to isolated DNA molecules encoding the proteinaceous molecules described above (see, for example, SEQ ID NO:1).

Other aspects of the present invention relate to recombinant expression vectors capable of transferring the recited polynucleotide molecules to suitable host cells, and to cells transformed by these expression vectors.

In another aspect the present invention relates to methods of producing the recited proteinaceous molecules by recombinant means.

In a further aspect of the present invention, it relates to pharmaceutical compositions comprising the recited proteinaceous molecules.

In yet another aspect, the present invention relates to specific binding partners to the recited proteinaceous molecules.

In still another aspect, the present invention relates to methods of discovering ligands for canine PTH1.

The above aspects of the present invention enable production of large quantities of proteinaceous molecules having canine PTH1 activity. It is contemplated that such molecules can be used, e.g., for veterinary purposes to treat dogs, as described in greater detail hereinbelow.

### DESCRIPTION OF THE FIGURE SHEETS

FIG. 1 provides the DNA sequence of canine PTH1 cDNA. A canine kidney library was screened using the human PTH1 coding region DNA as probe, resulting in identification of the canine PTH1 cDNA. Nucleotides corresponding to the coding region of canine PTH1 cDNA and the putative amino acids are displayed as capital letters. Lower case letters are used to depict the nucleotides of the 5′ and 3′ untranslated regions of the cDNA. Putative transmembrane spanning segments of the PTH1 protein are boxed and sequentially numbered with Roman numerals. The solid black bar indicates the peptide to which the anti-PTH1 antibody was generated. The nucleotide sequence of canine PTH1 cDNA has been deposited in GenBank with the accession number AF167095.

FIG. 2 provides a comparison of the PTH1 cDNA protein coding regions from canine, rat, mouse, and human. Boxed regions indicate areas of 100% nucleotide identity between the four species. Alignment of the PTH1 protein coding regions was accomplished using GeneWorks (Oxford Molecular Group, Campbell, Calif.).

FIG. 3 provides a comparison of the putative proteins encoded by canine, rat, mouse, and human PTH1 cDNAs. Boxed regions indicate areas of 100% amino acid identity between the four species. Alignment of the PTH1 proteins was accomplished using GeneWorks.

FIG. 4 provides a competitive binding analysis of canine PTH1 transfected CHO cells. Canine PTH1/pCR3.1 transiently transfected CHO cells exhibited high affinity binding of human PTH (1-34), canine PTH (1-34), and canine/human PTHrP (1-34). The similarly sized human aldosterone inhibitory factor (ASIF, 1-35) did not bind to the transfected canine PTH1. Mock transfected cells did not bind any of the peptides.

FIG. 5 provides a graph demonstrating canine PTH1 activation and secondary signaling. CHO cells transiently transfected with canine PTH1 were exposed to the peptides indicated (10⁻⁷ M) and subsequently assayed for intracellular cAMP accumulation. The non-specific peptide, aldosterone secretion inhibitory factor (ASIF, 1-35) that demonstrated no binding to canine PTH1 (FIG. 4), did not activate this receptor. Exposure of the transfected cells to canine PTH and PTHrP (dPTH and dPTHrP) and human PTH (hPTH) resulted in PTH1 activation as demonstrated by an increase in intracellular cAMP. Non-transfected cells exhibited no increase in cAMP following treatment with any peptide.

### DETAILED DESCRIPTION OF THE INVENTION

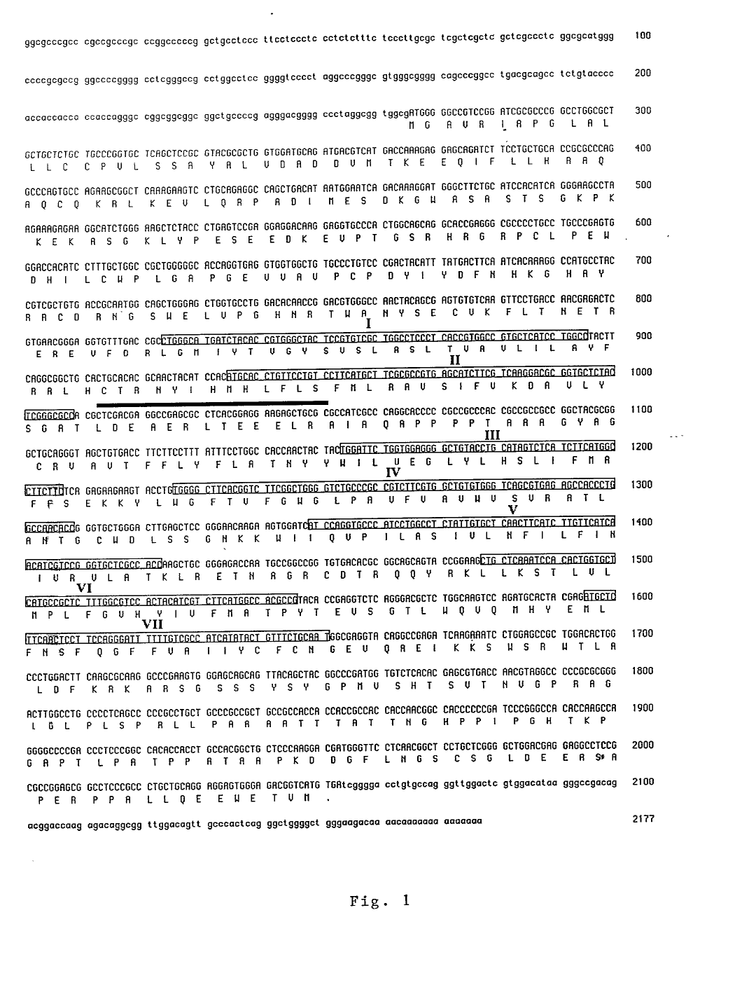

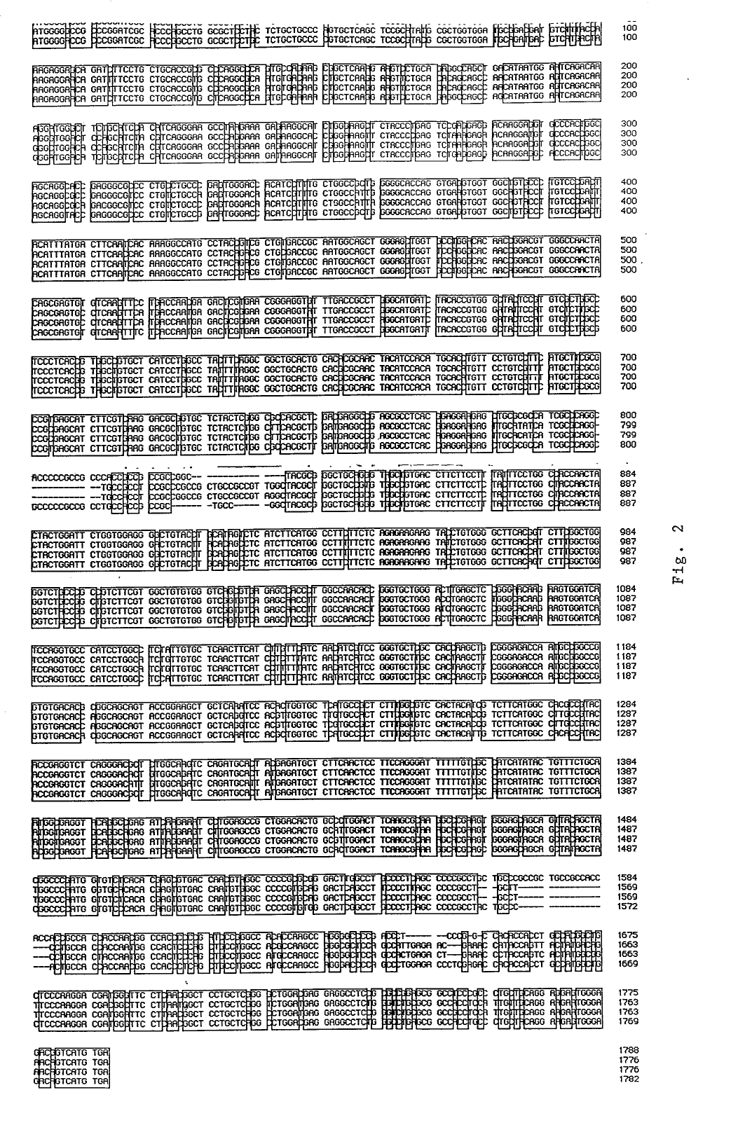

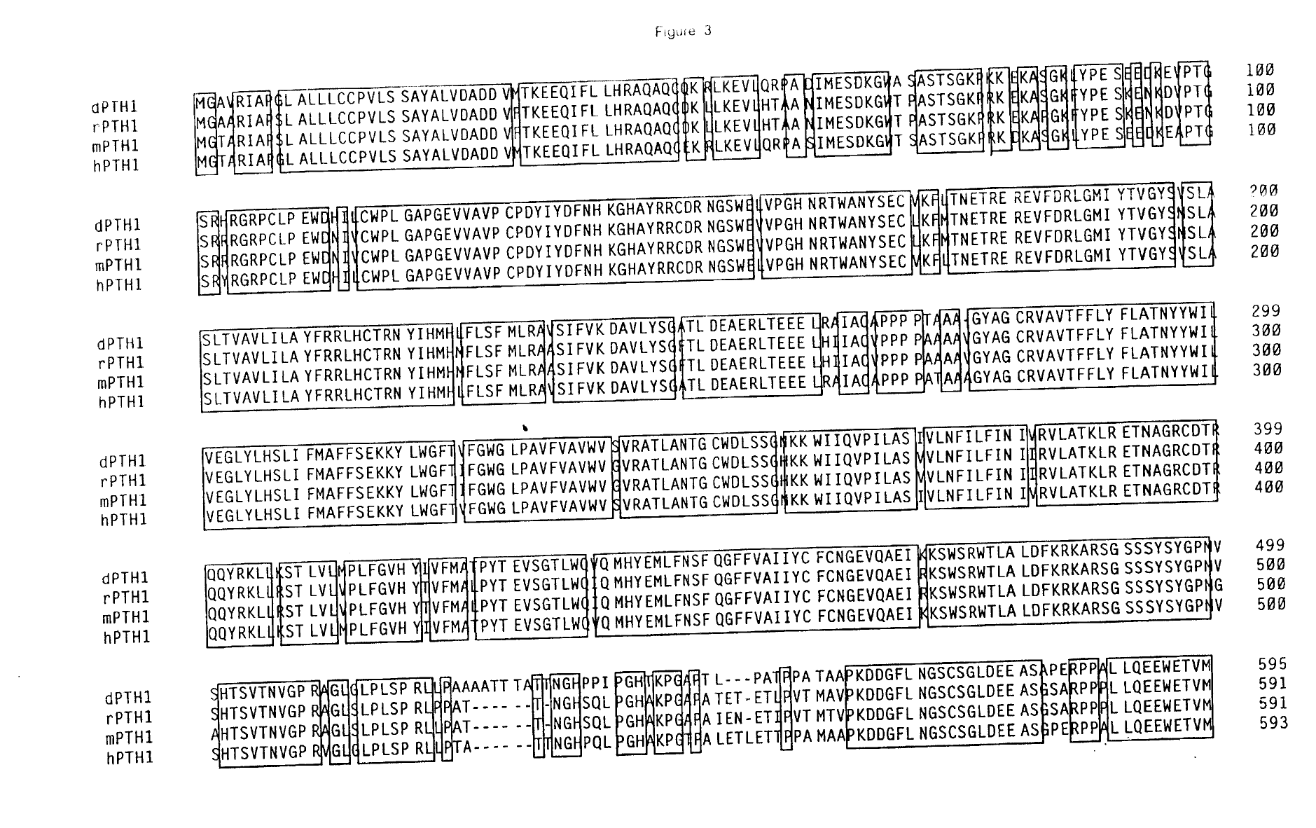

Screening of a canine kidney cDNA library (1×10⁶ clones) using a probe corresponding to the complete protein coding region of human PTH1 resulted in the identification of approximately 1200 positive plaques (0.12%). Of these positive plaques, 12 were randomly selected and subjected to two consecutive additional rounds of plaque purification. Following conversion of the 12 plaque purified clones from λTriplEx to pTriplEx, clone size was determined by performing restriction analysis on plasmid DNAs. Two clones with inserts large enough to encompass the complete canine PTH1 cDNA were fully sequenced. As shown in FIGS. 1, 2 and 3, the sequence of the canine PTH1 cDNA revealed 7 potential transmembrane regions in a protein coding region with >91% identity to human PTH1 at the nucleotide level and >95% identity at the predict amino acid level.

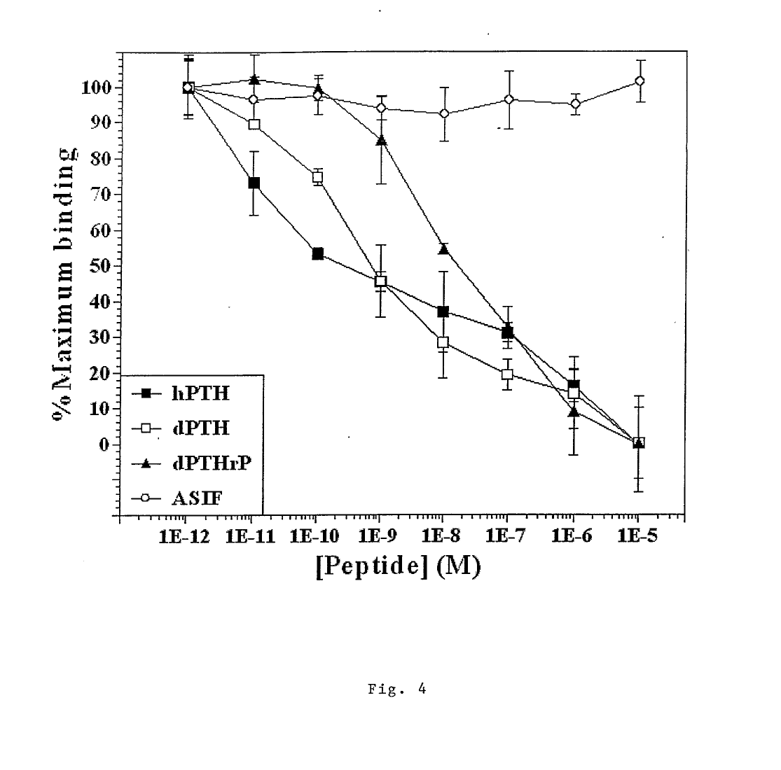

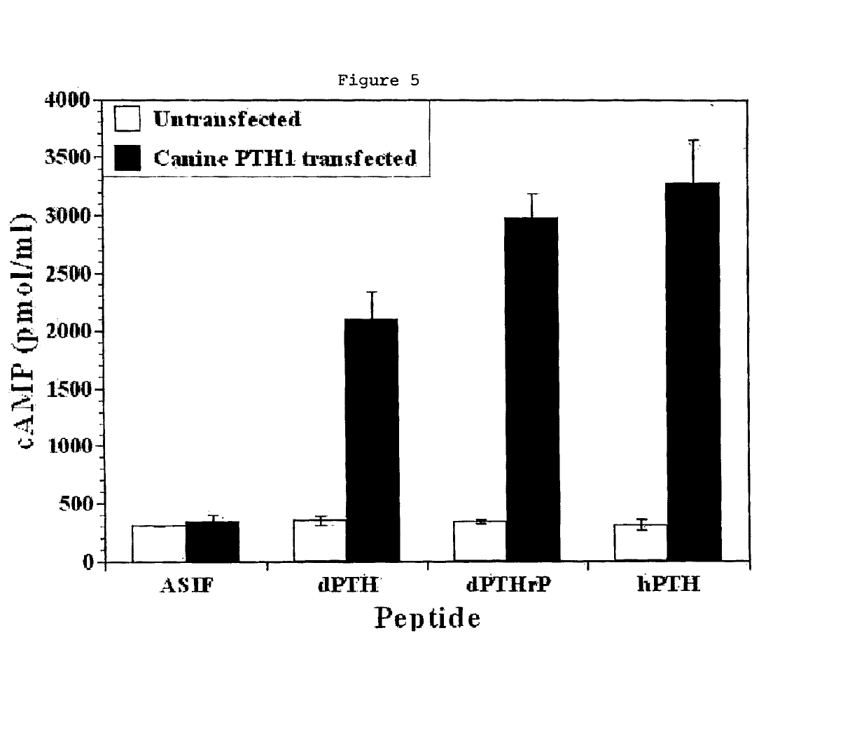

To test the binding and signaling characteristics of this putative canine PTH receptor, the open reading frame was cloned into the mammalian expression vector pCR3.1 and transiently transfected into CHO cells. Human and canine PTH, as well as canine PTHrP peptides, demonstrated specific binding to the cloned canine PTH1 receptor (FIG. 4). IC₅₀s values for these peptides were similar to values previously reported against PTH1 cloned from other species. Binding of these peptides to CHO cells transfected with PTH1 also resulted in elevated intracellular cAMP levels (FIG. 5). Conversely, human aldosterone secretion inhibitory factor (1-35), a similarly sized peptide, did not bind or activate the cloned canine PTH1 (FIGS. 4 and 5).

Canine multiple tissue Northern analysis of poly-A⁺ RNAs demonstrated the expected high levels of PTH1 mRNA in kidney with significantly less signal detectable in aorta, heart and lung. Upon overexposure of the blot, PTH1 hybridization signal was also detectable, albeit at low levels, in prostate, skeletal muscle and testis. The size of the canine PTH1 transcript identified by this analysis was approximately 2400 bases. 5 μg of canine tissue poly A⁺ RNAs were fractionated on a 0.9% agarose-1.89% formaldehyde gel, blotted to Nytran and probed with a ³²P-labeled DNA segment containing the human PTH1 coding region. Hybridization signal was detected using phosphorimage analysis. All tissues examined demonstrated detectable PTH1 mRNA at approximately 2400 bp.

Immunohistochemical localization of PTH1 protein in canine kidney and bone tissue samples resulted in the identification of specific immunoreactivity on both the endosteal and periosteal surfaces of the calvarium as well as in the periosteal layers of the femur and tibia immediately adjacent to newly formed bone. As expected, growth plate chondrocytes, the outer layers of the kidney cortex, and the collecting tubes in the kidney medulla were also positive for PTH1 immunoreactivity. Anti-PTH1 antibodies generated against the peptide CTLDEAERLTEEELH (BabCo, Richmond, Calif.) were used to localize PTH1 protein in canine kidney and bone.

A) Calvarium: Both periosteal and endosteal surfaces show specific staining. Endosteal micrographs indicate staining of the endosteal surface as well as osteocytes. Periosteal micrographs show staining of periosteal cells adjacent to the bone surface, as well as staining of overlying muscle. No staining is observed in controls lacking primary antibody.

B) Proximal tibia: No staining for PTH1 is observed in cortical bone while the cells of the periosteal layer adjacent to the bone surface are heavily labeled. Chondrocytes throughout the growth plate are specifically stained.

C) Distal femur: Cells of the periosteal layer adjacent to the bone surface are heavily labeled as are cells (probably osteoblasts) lining the trabecular bone. Cells deep in cortical bone are not labeled but osteocytes in areas adjacent to the active trabecular osteoblasts are positive for PTH1.

D) Kidney: specific PTH1 immunostaining is observed in the outer layer of the cortex and in the collecting tubules of the medulla. No staining is observed in controls lacking primary antibody.

### Definitions

“Proteinaceous molecule” generally encompasses any molecule made up of a plurality of amino acids. The term is broad enough to include peptides, oligopeptides, and proteins. Typically, the amino acids in the proteinaceous material will be selected from the 20 naturally occurring amino acids. However, amino acid analogs and derivatives could also be included in the proteinaceous molecule. The proteinaceous molecule will usually be made up of about the same number of amino acids contained in naturally occurring caPTH1 (i.e., about 595 amino acids). In addition, however, the proteinaceous molecule may have a greater or lesser number of amino acids, as long as the molecules retain an activity of caPTH1. This activity need not be quantitatively the same as the activity of the natural protein, and may be more or less than that activity, as long as it is measurable by an assay of caPTH1 activity (see below). Preferably, such molecules will possess at least about 50% of the activity of the naturally occurring caPTH1. In some hosts, expression of recombinant caPTH1 will result in a protein having an N-terminal methionine residue. Amino acid sequences containing such a residue on the N-terminus thereof are also within the scope of the present invention.

By “conservative substitution” is meant a substitution, addition, or deletion of an amino acid in a proteinaceous molecule that is not expected to significantly affect the activity of thereof. For example, the replacement of one hydrophobic amino acid for another in a transmembrane region of a proteinaceous molecule will seldom have any significant impact on the activity of thereof. Other conservative substitutions are well known to those skilled in the art.

By “activity of caPTH1” is meant any activity that is measurable by an in vivo or in vitro caPTH1 assay. Qualitatively, the activity will generally be one that is possessed by the naturally occurring caPTH1 protein. Cells expressing, or thought to be expressing, caPTH1 protein may be assayed for both the levels of caPTH1 receptor activity and levels of caPTH1 protein. Assessing caPTH1 receptor activity preferably involves the direct introduction of a labelled ligand to the cells and determining the amount of specific binding of the ligand to the caPTH1-expressing cells. Binding assays for receptor activity are known in the art (Sando et al., Biochem. Biophys. Res. Comm., 200:1329-1333 (1994)). Specific assays for PTH receptor acticity are described at columns 9-12 in U.S. Pat. No. 5,494,806 (the entirety of which is incorporated by reference herein). Levels of caPTH1 protein in host cells may be quantitated by a variety of techniques including, but not limited to, immunoaffinity and/or ligand affinity techniques. caPTH1-specific affinity beads or caPTH 1-specific antibodies are used to isolate ³⁵S-methionine labelled or unlabelled caPTH1 protein. Labelled caPTH1 protein may be analyzed by SDS-PAGE. Unlabelled caPTH1 protein may be detected by Western blotting, ELISA, or RIA assays employing caPTH 1-specific antibodies.

By “isolated” is meant that a substance is substantially free from normally occurring impurities or other molecules, especially other proteinaceous molecules, salts, other cellular constituents, and the like. Isolation may typically be carried out by standard methods in the art of nucleotide, peptide, and/or protein purification or synthesis. Following expression of caPTH1 in a host cell, caPTH1 protein may be recovered to provide caPTH1 in active form, capable of binding caPTH1-specific ligands. Several caPTH1 purification procedures are available and suitable for use. Recombinant caPTH1 may be purified from cell lysates and extracts, or from conditioned culture medium, by various combinations of, or individual application of salt fractionation, ion exchange chromatography, size exclusion chromatography, hydroxylapatite adsorption chromatography, and hydrophobic interaction chromatography. In addition, recombinant caPTH1 can be separated from other cellular proteins by use of an immunoaffinity column made with monoclonal or polyclonal antibodies specific for full length nascent caPTH1, or polypeptide fragments of caPTH1.

“Polynucleotide sequences” encompass both DNA- and RNA-containing molecules. The DNA molecules will preferably be intronless sequences (i.e., cDNA), but can contain enhancer sequences, termination sequences, and the like, to facilitate or increase expression in a particular host. mRNA molecules are also encompassed by the term.

By “recombinant expression vector” is meant a vector (e.g., a plasmid or λphage) that is capable of transferring polynucleotide sequences contained therein into cells of a host organism for expression of the transferred sequences. The sequences are operably linked to other sequences capable of effecting or modulating their expression. Such expression vectors must be replicable in a host organism. For example, any DNA sequence that is capable of effecting expression of a specified DNA sequence disposed therein is included in this term as it is applied to the specified sequence.

The “cells” that may be transformed by way of the vectors described above are those that are capable of expressing the polynucleotide sequences that have been transferred by the vector. Culturing conditions for such cells may be those standard in the art of recombinant protein production.

“Specific binding partners” are molecules that are capable, on a molecular level, of recognizing and interacting with the proteinaceous or polynucleotide molecules described herein. Included within this term are immunological binding partners such as antibody molecules, antigen-binding fragments of antibodies (e.g., Fab and F(ab+)₂ fragments), single chain antigen-binding molecules, and the like, whether produced by hybridoma or rDNA technology. Other proteinaceous or non-proteinaceous binding partners are also included within the broad term.

### Proteinaceous Molecules

The proteinaceous molecules of this invention will now be described in greater detail. SEQ ID NO:2 corresponds to the naturally occurring canine peptide having 595 amino acids.

The family of caPTH1 proteins provided herein also includes proteinaceous molecules in which one or more of the amino acids in the above-recited amino acid sequences has been deleted, modified, or changed to another amino acid. Site directed mutagenesis is a preferred technique enabling conversion of one amino acid to another. For example, one or more of the cysteine residues may be changed to another amino acid such as serine. One possible reason for such a change would be to eliminate one or more unwanted disulfide bonds. See, for example, U.S. Pat. No. 4,518,584.

Other contemplated specific changes in the natural amino acid sequences involve modification of any asparagine glycosylation sites. Modification of asparagine can eliminate glycosylation at the modified site. Thus, for example, the asparagine could be changed to a glutamine, thereby eliminating glycosylation at the site. See, for example, Miyajima et al., EMBO J., 5(6):1993 (1986).

To determine the caPTH1 cDNA sequence(s) that yields optimal levels of receptor activity and/or caPTH1 protein, caPTH1 cDNA molecules including but not limited to the following can be constructed: the full-length open reading frame of the caPTH1 cDNA and various constructs containing portions of the cDNA encoding only specific domains of the receptor protein or rearranged domains of the protein. All constructs can be designed to contain none, all, or portions of the 5′ and/or 3′ untranslated region of caPTH1 cDNA. caPTH1 activity and levels of protein expression can be determined following the introduction, both singly and in combination, of these constructs into appropriate host cells. Following determination of the caPTH1 cDNA cassette yielding optimal expression in transient assays, this caPTH1 cDNA construct is transferred to a variety of expression vectors (including recombinant viruses), including but not limited to those for mammalian cells, plant cells, insect cells, oocytes, E. coli, and yeast cells.

The modifications of the amino acid sequences described above that form a part of the present invention are those that result in proteinaceous molecules having an activity of canine PTH1. Such activity may be determined using the assays described herein, or equivalent assays now known or yet to be developed. The family of proteinaceous molecules of the present invention will also generally be highly homologous to SEQ ID NO:2, which means that several of the amino acids in each sequence may be deleted, modified, or changed (assuming the resulting proteinaceous material retains at least some activity in common with canine PTH1).

The proteinaceous molecules of the present invention may further be labeled by attachment to a detectable marker substance (e.g., radiolabeled with ¹²⁵I ) to provide reagents useful in vitro or in vivo.

### Polynucleotide Sequences

The present invention is also directed to polynucleotide sequences. Preferred polynucleotide sequences are DNA molecules that encode the proteinaceous molecules of the present invention, described above. A preferred DNA sequence (SEQ ID NO:1) is the one shown in FIG. 1 herein. The novel cDNA sequence illustrated in FIG. 1 included in a plasmid has been deposited before the filing date in GenBank under accession number AF1 67095. The cDNA of FIG. 1 is described in greater detail herein.

It is to be recognized that more than one DNA sequence can encode the same amino acid sequence, due to the degeneracy of the genetic code. All such sequences are encompassed by the polynucleotide sequences described herein.

The presently preferred method of obtaining the cDNA of FIG. 1 is described herein. However, any of a variety of alternative procedures may be used to clone caPTH1 cDNA. These methods include, but are not limited to, direct functional expression of the caPTH1 cDNA following the construction of an caPTH1-containing cDNA library in an appropriate expression vector system. Another method is to screen an caPTH1-containing cDNA library constructed in a bacteriophage or plasmid shuttle vector with a labelled oligonucleotide probe designed from the amino acid sequence of the caPTH1 protein. The preferred method consists of screening an caPTH1-containing cDNA library constructed in a bacteriophage or plasmid shuttle vector with a partial cDNA encoding the caPTH1 protein. This partial cDNA is obtained by the specific PCR amplification of caPTH1 DNA fragments through the design of degenerate oligonucleotide primers from the amino acid sequence known for other G protein-coupled receptors which are related to the caPTH1 receptors.

It is readily apparent to those skilled in the art that other types of libraries, as well as libraries constructed from other cells or cell types, may be useful for isolating caPTH1-encoding DNA. Other types of libraries include, but are not limited to, cDNA libraries derived from other cells or cell lines other than canine kidney cells, and genomic DNA libraries.

It is readily apparent to those skilled in the art that suitable cDNA libraries may be prepared from cells or cell lines that have caPTH1 activity. The selection of cells or cell lines for use in preparing a cDNA library to isolate caPTH1 cDNA may be done by first measuring cell associated caPTH1 activity using the known labelled ligand binding assay cited herein.

Preparation of cDNA libraries can be performed by standard techniques well known in the art. Well known cDNA library construction techniques can be found for example, in Maniatis et al., Molecular Cloning: A Laboratory Manual (Cold Spring Harbor Laboratory, Cold Spring Harbor, N.Y., 1982).

It is also readily apparent to those skilled in the art that DNA encoding caPTH1 may also be isolated from a suitable genomic DNA library. Construction of genomic DNA libraries can be performed by standard techniques well known in the art. Well known genomic DNA library construction techniques can be found in Maniatis et al., Molecular Cloning: A Laboratory Manual (Cold Spring Harbor Laboratory, Cold Spring Harbor, N.Y., 1982).

In order to clone the caPTH1 gene by one of the preferred methods, the amino acid sequence or DNA sequence of caPTH1 or a homologous protein is necessary. To accomplish this, caPTH1 protein or a homologous protein may be purified and partial amino acid sequence determined by automated sequenators. It is not necessary to determine the entire amino acid sequence, but the linear sequence of two regions of 6 to 8 amino acids can be determined for the PCR amplification of a partial caPTH1 DNA fragment.

Once suitable amino acid sequences have been identified, the DNA sequences capable of encoding them are synthesized. Because the genetic code is degenerate, more than one codon may be used to encode a particular amino acid, and therefore, the amino acid sequence can be encoded by any of a set of similar DNA oligonucleotides. Only one member of the set will be identical to the caPTH1 sequence but others in the set may be capable of hybridizing to caPTH1 DNA even though they contain mismatches. The mismatched DNA oligonucleotides may still sufficiently hybridize to the caPTH1 DNA to permit identification and isolation of caPTH1 encoding DNA.

Using one of the preferred methods, cDNA clones encoding caPTH1 are isolated in a two-stage approach employing polymerase chain reaction (PCR) based technology and cDNA library screening. In the first stage, NH₂-terminal and internal amino acid sequence information from the purified caPTH1 or a homologous protein is used to design degenerate oligonucleotide primers for the amplification of caPTH1-specific DNA fragments. In the second stage, these fragments are cloned to serve as probes for the isolation of full length cDNA from a cDNA library derived from canine kidney cells.

Polynucleotide products of the present invention may be labeled with detectable markers (such as radiolabels and non-isotopic labels such as biotin) and employed, for example, in DNA hybridization processes to locate the canine gene position and/or the position of any related gene family in a chromosomal map. They may also be used for identifying canine gene disorders at the DNA level and used as gene markers for identifying neighboring genes and their disorders.

### Expression Vectors, Hosts, and Recombinant Methods

The cloned caPTH1 cDNA obtained through the methods described herein may be recombinantly expressed by molecular cloning into an expression vector containing a suitable promoter and other appropriate transcription regulatory elements, and transferred into prokaryotic or eukaryotic host cells to produce recombinant caPTH1. Techniques for such manipulations can be found described in Maniatis et al., supra, and are well known in the art.

Expression vectors are defined herein as DNA sequences that are required for the transcription of cloned DNA and the translation of their mRNAs in an appropriate host. Such vectors can be used to express eukaryotic DNA in a variety of hosts such as bacteria, bluegreen algae, plant cells, insect cells, and animal cells.

Specifically designed vectors allow the shuttling of DNA between hosts such as bacteria-yeast or bacteria-animal cells. An appropriately constructed expression vector preferably contains: an origin of replication for autonomous replication in host cells, selectable markers, a limited number of useful restriction enzyme sites, a potential for high copy number, and active promoters. A promoter is defined as a DNA sequence that directs RNA polymerase to bind to DNA and initiate RNA synthesis. A strong promoter is one that causes mRNAs to be initiated at high frequency. Expression vectors may include, but are not limited to, cloning vectors, modified cloning vectors, specifically designed plasmids, or viruses.

A variety of mammalian expression vectors may be used to express recombinant caPTH1 in mammalian cells. Commercially available mammalian expression vectors that may be suitable for recombinant caPTH1 expression, include but are not limited to, pMClneo (Stratagene), pXT1 (Stratagene), pSG5 (Stratagene), pcDNAl, pcDNAlamp (Invitrogen), EBO-pSV2-neo (ATCC 37593) pBPV-1(8-2) (ATCC 37110), pdBPV-MMTneo(342-12) (ATCC 37224) pRS-Vgpt (ATCC 37199), pRSVneo (ATCC 37198), pSV2-dhfr (ATCC 37146), pUCTag (ATCC 37460), and IZD35 (ATCC 37565)

DNA encoding caPTH1 may also be cloned into an expression vector for expression in a host cell. Host cells may be prokaryotic or eukaryotic, including but not limited to bacteria, yeast, mammalian cells including but not limited to cell lines of human, bovine, porcine, monkey, and rodent origin, and insect cells including but not limited to drosophila derived cell lines. Cell lines derived from mammalian species which may be suitable and which are commercially available, include but are not limited to, CV-1 (ATCC CCL 70), COS-1 (ATCC CRL 1650), COS-7 (ATCC CRL 1651), CHO-Kl (ATCC CCL 61), 3T3 (ATCC CCL 92), NIHi3T3 (ATCC CRL 1658), HeLa (ATCC CCL 2), CI271 (ATCC CRL 1616), BS-C-1 (ATCC CCL 26), and MRC-5 (ATCC CCL 171).

The expression vector may be introduced into host cells via any one of a number of techniques including but not limited to transformation, transfection, protoplast fusion, and electroporation. The expression vector-containing cells are individually analyzed to determine whether they produce caPTH1 protein. Identification of caPTH1 expressing cells may be done by several means, including but not limited to immunological reactivity with anti-caPTH1 antibodies, and the presence of host cell-associated caPTH1 activity.

Expression of caPTH1 DNA may also be performed using in vitro produced synthetic mRNA. Synthetic mRNA can be efficiently translated in various cell-free systems, including but not limited to wheat germ extracts and reticulocyte extracts, as well as efficiently translated in cell based systems, including but not limited to microinjection into frog oocytes, with microinjection into frog oocytes being preferred.

A variety of prokaryotic cells known to those of ordinary skill in this art may be utilized. A few exemplary strains include E. coli, Bacillus subtilis, and various strains of Pseudomonas.

In addition to bacteria, eukaryotic microbes, such as yeast, may also be used as a host. Laboratory strains of Saccharomyces cerevisiae are most commonly used.

It is also possible to express genes encoding polypeptides in eukaryotic host cell cultures derived from multi-cellular organisms. Useful host cell lines include Vero, HeLa, COS, and Chinese hamster ovary (CHO) cells. Expression vectors for such cells ordinarily include promoters and control sequences compatible with mammalian cells such as, for example, the commonly used early and later promoters from Simian Virus 40 (SV40) or other viral promoters such as those derived from polyoma, adenovirus 2, bovine papilloma virus, avian sarcoma viruses, immunoglobulin promoters, and heat shock promoters. Enhancer regions may also be included as desired.

Other examples of hosts, vectors, enhancers, promoters, etc., may be found in the following exemplary U.S. Pat. Nos. 4,810,643; 4,766,075; and 4,847,201, each of which is incorporated by reference herein.

### Specific Binding Partners

Specific binding partners directed to the proteinaceous molecules and polypeptides of the present invention may be generated by any standard technique known to those of skill in the art. Preferred specific binding partners are immunological binding partners such as intact antibodies and fragments thereof. The immunological binding partners are preferably monoclonal antibodies directed to a specific antigen, which are prepared by standard techniques of monoclonal antibody production.

These specific binding partners may be utilized, for example, to purify the proteinaceous materials or polynucleotides of the present invention. Specific binding partners in labeled form may be utilized to indicate the presence of the proteinaceous molecules or polynucleotides of the present invention. In one preferred embodiment, a specific binding partner of a polynucleotide of the present invention may be utilized in labeled form to locate the natural gene on a chromosome.

Monospecific antibodies to caPTH1 are purified from mammalian antisera containing antibodies reactive against caPTH1 or are prepared as monoclonal antibodies reactive with caPTH1 using the technique of Kohler and Milstein, Nature, 256:495-497 (1975). Monospecific antibody as used herein is defined as a single antibody species or multiple antibody species with homogenous binding characteristics for caPTH1. Homogenous binding as used herein refers to the ability of the antibody species to bind to a specific antigen or epitope, such as those associated with the caPTH1, as described above. caPTH1 specific antibodies are raised by immunizing animals such as mice, rats, guinea pigs, rabbits, goats, horses, and the like, with an appropriate concentration of caPTH1 either with or without an immune adjuvant.

Preimmune serum is collected prior to the first immunization. Each animal receives between about 0.1 μg and about 1000 μg of caPTH1 associated with an acceptable immune adjuvant. Such acceptable adjuvants include, but are not limited to, Freund's complete, Freund's incomplete, alum-precipitate, water in oil emulsion containing Corynebacterium parvum, and tRNA. The initial immunization consists of the enzyme in, preferably, Freund's complete adjuvant at multiple sites either subcutaneously (SC), intraperitoneally (IP), or both. Each animal is bled at regular intervals, preferably weekly, to determine antibody titer. The animals may or may not receive booster injections following the initial immunization. Those animals receiving booster injections are generally given an equal amount of caPTH1 in Freund's incomplete adjuvant by the same route. Booster injections are given at about three week intervals until maximal titers are obtained. At about 7 days after each booster immunization or about weekly after a single immunization, the animals are bled, the serum collected, and aliquots are stored at about −20° C.

Monoclonal antibodies (mAb) reactive with caPTH1 are prepared by immunizing inbred mice, preferably Balb/c, with caPTH1. The mice are immunized by the IP or SC route with about 1 μg to about 100 μg, preferably about 10 μg, of caPTH1 in about 0.5 ml buffer or saline incorporated in an equal volume of an acceptable adjuvant, as discussed above. Freund's complete adjuvant is preferred. The mice receive an initial immunization on day 0 and are rested for about 3 to about 30 weeks. Immunized mice are given one or more booster immunizations of about 1 to about 100 μg of caPTH1 in a buffer solution such as phosphate buffered saline by the intravenous (IV) route. Lymphocytes, from antibody positive mice, preferably splenic lymphocytes, are obtained by removing spleens from immunized mice by standard procedures known in the art. Hybridoma cells are produced by mixing the splenic lymphocytes with an appropriate fusion partner, preferably myeloma cells, under conditions which will allow the formation of stable hybridomas. Fusion partners may include, but are not limited to: mouse myelomas P3/NS1/Ag 4-1, MPC-11, S-194, and Sp 2/0, with Sp 2/0 being preferred. The antibody producing cells and myeloma cells are fused in polyethylene glycol, about 1000 mol. wt., at concentrations from about 30% to about 50%. Fused hybridoma cells are selected by growth in hypoxanthine, thymidine, and aminopterin supplemented Dulbecco's Modified Eagles Medium (DMEM) by procedures known in the art. Supernatant fluids are collected from growth positive wells on about days 14, 18, and 21 and are screened for antibody production by an immunoassay such as solid phase immunoradioassay (SPIRA) using caPTH1 as the antigen. The culture fluids are also tested in the Ouchterlony precipitation assay to determine the isotype of the mAb. Hybridoma cells from antibody positive wells are cloned by a technique such as the soft agar technique of MacPherson, (Soft Agar Techniques, in Tissue Culture Methods and Applications, IQ-use and Paterson, Eds., Academic Press, 1973).

Monoclonal antibodies are produced in vivo by injection of pristine primed Balb/c mice, approximately 0.5 ml per mouse, with about 2×10⁶ to about 6×10⁶ hybridoma cells about 4 days after priming. Ascites fluid is collected at approximately 8-12 days after cell transfer and the monoclonal antibodies are purified by techniques known in the art.

In vitro production of anti-caPTH1 mAb is carried out by growing the hydridoma in DMEM containing about 2% fetal calf serum to obtain sufficient quantities of the specific mAb. The mAb are purified by techniques known in the art.

Antibody titers of ascites or hybridoma culture fluids are determined by various serological or immunological assays which include, but are not limited to, precipitation, passive agglutination, enzyme-linked immunosorbent antibody (ELISA) technique, and radioimmunoassay (RIA) techniques. Similar assays are used to detect the presence of caPTH1 in body fluids or tissue and cell extracts.

It is readily apparent to those skilled in the art that the above described methods for producing monospecific antibodies may be utilized to produce antibodies specific for caPTH1 polypeptide fragments or full-length caPTH1 polypeptide.

caPTH1 antibody affinity columns are made by adding the antibodies to Affigel-10 (Biorad), a gel support that is pre-activated with N-hydroxysuccinimide esters such that the antibodies form covalent linkages with the agarose gel bead support. The antibodies are then coupled to the gel via amide bonds with the spacer arm. The remaining activated esters are then quenched with 1 M ethanolamine HCl (pH 8). The column is washed with water followed by 0.23M glycine HCl (pH 2.6) to remove any non-conjugated antibody or extraneous protein. The column is then equilibrated in phosphate buffered saline (pH 7.3) and the cell culture supernatants or cell extracts containing caPTH1 or caPTH1 fragments are slowly passed through the column. The column is then washed with phosphate buffered saline until the optical density (A₂₈₀) falls to background, then the protein is eluted with 0.23M glycine-HCl (pH 2.6). The purified caPTH1 protein is then dialyzed against phosphate buffered saline.

### Screening For PTH Receptor Antagonists And Agonists

The polypeptides and antibodies of the invention and other compounds may be screened for PTH-competition and for antagonistic or agonistic properties using assays described herein.

In one example, those antibodies that recognize the PTH receptor on the intact cells are screened for their ability to compete with PTH or PTHrP for binding to a PTH/PTHrP receptor. Cells expressing PTH receptor on the cell surface are incubated with the ¹²⁵I-PTH analog, ¹²⁵I-NIePTH, or ¹²⁵I-PTHrP in the presence or absence of the polyclonal or monoclonal antibody to be tested, for 4 h at 15° C. The antibody used may be from crude antiserum, cell medium, or ascites, or in purified form. After incubation, the cells are rinsed with binding buffer (e.g., physiological saline), lysed, and quantitatively analyzed for radioactivity using a gamma-counter. Antibodies that reduce binding of the PTH analog to the PTH receptor are classified as competitive; those which do not are noncompetitive.

Compounds, including small molecules, antibodies, and polypeptides, may be screened for their agonistic or antagonistic properties using the cAMP accumulation, intracellular calcium, and/or inositol phosphate assays described in U.S. Pat. No. 5,494,806, as well as other assays. Cells expressing PTH receptor on the cell surface are incubated with PTH, PTH-receptor antibody, or a combination of both, for 5-60 minutes at 37° C., in the presence of 2 mM IBMX (3-isobutyl-1-methyl-xanthine, Sigma, St. Louis, Mo.). Cyclic AMP accumulation is measured by specific radioimmunoassay, as described above. A compound that competes with PTH for binding to the PTH receptor, and that inhibits the effect of PTH on cAMP accumulation, is considered a competitive PTH antagonist. Conversely, a compound that does not compete for PTH binding to the PTH receptor, but which still prevents PTH activation of cAMP accumulation (presumably by blocking the receptor activation site) is considered a non-competitive antagonist. A compound that competes with PTH for binding to the PTH receptor, and which stimulates cAMP accumulation in the presence or absence of PTH, is a competitive agonist. A compound that does not compete with PTH for binding to the PTH receptor but which is still capable of stimulating cAMP accumulation in the presence or absence of PTH, or which stimulates a higher accumulation than that observed by PTH alone, would be considered a non-competitive agonist.

The canine PTH1 receptor of the present invention may be obtained from both native and recombinant sources for use in an assay procedure to identify receptor modulators. In general, an assay procedure to identify canine PTH1 receptor modulators will contain the canine PTH1 receptor of the present invention, and a test compound or sample which contains a putative canine PTH1 receptor modulator. The test compounds or samples may be tested directly on, for example, purified receptor protein whether native or recombinant, subcellular fractions of receptor-producing cells whether native or recombinant, and/or whole cells expressing the receptor whether native or recombinant. The test compound or sample may be added to the receptor in the presence or absence of a known labelled or unlabelled receptor ligand. The modulating activity of the test compound or sample may be determined by, for example, analyzing the ability of the test compound or sample to bind to the receptor, activate the receptor, inhibit receptor activity, inhibit or enhance the binding of other compounds to the receptor, modifying receptor regulation, or modifying an intracellular activity.

The identification of modulators of caPTH1 receptor activity are useful in treating disease states involving the caPTH1 receptor activity. Other compounds may be useful for stimulating or inhibiting activity of the receptor. These compounds could be useful as means of stimulating or inhibiting bone formation. Such compounds could be of use in the treatment of diseases in which activation of the caPTH1 receptor results in either cellular proliferation, induction of cellular neoplastic transformations, or metastatic tumor growth, and hence could be used in the prevention and/or treatment of cancers. The isolation and purification of an caPTH1-encoding DNA molecule would be useful for establishing the tissue distribution of caPTH1 receptors as well as establishing a process for identifying compounds which modulate caPTH1 receptor activity.

### Uses

The polypeptides, antibodies, and other compounds of the invention are useful for the diagnosis, classification, prognosis, and/or treatment of canine disorders which may be characterized as related to the interaction between a cell receptor of the invention and its specific ligand. For example, some forms of hypercalcemia and hypocalcemia are related to the interaction between PTH and PTHrP and the PTH/PTHrP receptor(s). Hypercalcemia is an condition in which there is an abnormal elevation in serum calcium level; it is often associated with other diseases, including hyperparathyroidism, osteoporosis, carcinomas of the breast, lung and prostrate, epidermoid cancers of the head and neck of the esophagus, multiple myeloma, and hypernephroma. Hypocalcemia, a condition in which the serum calcium level is abnormally low, may result from a deficiency of effective PTH, e.g., following thyroid surgery.

In a first example, the compounds of the invention are used to manufacture diagnostic agents which are used as canine diagnostic tools to diagnose hypercalcemia and to distinguish between hypercalcemic conditions, i.e., to differentiate hypercalcemia mediated by PTH or PTHrP (e.g., hyperparathyroidism and humoral hypercalcemia of malignancy), from hypercalcemia associated with diseases which do not involve these factors (e.g., local osteolytic hypercalcemia mediated by the presence of metastatic tumor cells in direct contact with bone, and certain rare types of malignancy-related hypercalcemias mediated by an increase of humoral factors, such as osteoclast activating factor (interleukin), lymphotoxin, calcitriol, type E prostaglandins, and vitamin D-like sterols).

In one method of diagnosis, serum total and/or ionized calcium levels are measured by standard techniques before and after the administration of the PTH or PTHrP antagonists of the invention. PTH or PTHrP related hypercalcemias would be detectable as a decrease in serum calcium levels following administration of the antagonist of the invention. In contrast, for hypercalcemic conditions mediated by factors other than PTH or PTHrP, the serum calcium levels would remain unchanged even after administration of the antagonist.

Another diagnostic application of the invention permits measurement of the level of PTH or PTHrP in a biological sample in order to diagnose PTH or PTHrP related tumors, e.g., tumors which are associated with humoral hypercalcemia of malignancy, and for monitoring the levels of PTH or PTHrP during cancer therapy. This method involves assaying binding of the recombinant parathyroid hormone receptor of the invention to PTH or PTHrP present in a tissue sample, using the binding assay described herein. The level of binding may be determined directly (e.g., by using radioactively labelled PTH receptor, and assaying the radioactivity bound to endogenous PTH). Alternatively, binding of PTH receptor to the sample (e.g., a tissue section) may be followed by staining of the tissue sections with an antibody specific for the PTH receptor, using standard immunological techniques (Chin et al., Hybridoma 5:339, 1986).

In a third diagnostic approach, one could stably transfect cell lines (by the methods described in Ausubel et al., Current Protocols in Molecular Biology, Wiley Publishers, N.Y., 1987) with a PTH receptor gene linked to an appropriate promoter (e.g., the metallothionine promoter). Alternatively, the PTH/PTHrP receptor could be expressed from a eukaryotic vector, i.e., pcDNAl, and cotransfected with a mutant DHFR gene that will allow further gene amplification via methotrexate selection (Simonsen et al., Proc. Natl. Acad. Sci., 80:2495-2499, 1983). Such high-level expression of the gene produces an immortal cell line which is oversensitive to PTH or PTHrP. Such cells provide a particularly useful tool for detecting serum blood levels of PTH or PTHrP. Such a cell line may be used for diagnosis of conditions involving elevated PTH or PTHrP levels (e.g., those described above) or for conditions involving unusually low levels of PTH or PTHrP (e.g., those described above). Such a cell line is also useful for monitoring the regression or increase of PTH or PTHrP levels during therapy for hypercalcemia or hypocalcemia, respectively.

A canine that is suspected of being hypercalcemic may be treated using the compounds of the invention. Rapid intervention is important because symptoms may appear abruptly and, unless reversed, can be fatal. In one application, serum calcium levels are stabilized by an immediate course of treatment which includes antagonists of PTH or PTHrP. Such antagonists include the compounds of the invention which have been determined (by the assays described herein) to interfere with PTH receptor-mediated cell activation. To administer the antagonist, the appropriate antibody or peptide is used in the manufacture of a medicament, generally by being formulated in an appropriate carrier such as physiological saline, and administered intravenously, at a dosage that provides adequate competition for PTH or PTHrP binding to the PTH receptor (e.g., a dosage sufficient to lower the serum calcium level to appropriate levels). Typical dosage would be 1 ng to 10 mg of the antibody or peptide per kg body weight per day. Treatment may be repeated as necessary for long term maintenance of acceptable calcium levels. This may be necessary for acute treatment of an underlying disease condition triggering hypercalcemia; or it may used, e.g., for chronic treatment of conditions such as osteoporosis.

In another application, the compounds of the invention which have been characterized, according to the methods of the invention, to be agonists are used therapeutically to treat hypocalcemia: e.g., that resulting from the partial or complete surgical removal of the parathyroid glands. Agonists may be formulated in a suitable carrier (e.g., physiological saline) and are preferably administered intravenously in a dosage that causes a rise in serum calcium to an acceptable level. A useful dosage range would be 1 ng to 10 mg of the agonist per kg body weight per day. Treatment may be repeated as necessary to maintain suitable serum calcium levels; long term treatment may be necessary for patients who have undergone parathyroid gland removal.

The nucleic acids of the invention may also be used therapeutically. Oligonucleotides which are antisense to PTH receptor mRNA (or nucleic acid constructs which express RNA that is antisense to PTH receptor mRNA) may be utilized as an anticancer therapy. This approach is useful, e.g., for hypercalcemias resulting from a genomic rearrangement or amplification which increases the amount or activity of PTH receptor, PTH, or PTHrP. The method would involve introduction of the antisense oligonucleotide into the tumor cells in vivo. The antisense strand hybridizes with endogenous PTH receptor mRNA, interfering with translation of the protein, thereby reducing production of PTH receptor in such cells, and reducing PTH/PTHrP-associated neoplastic growth. Methods for antisense design and introduction into host cells are described, for example, in Weinberg et al., U.S. Pat. No. 4,740,463, herein incorporated by reference.

Nucleic acids of the invention which encode a PTH receptor may also be linked to a selected tissue-specific promoter and/or enhancer and the resultant hybrid gene introduced, by standard methods (e.g., as described by Leder et al., U.S. Pat. No. 4,736,866, herein incorporated by reference), into an animal embryo at an early developmental stage (e.g., the fertilized oocyte stage), to produce a transgenic animal which expresses elevated levels of PTH receptor in selected tissues (e.g., the osteocalcin promoter for bone). Such promoters are used to direct tissue-specific expression of the PTH receptor in the transgenic animal. The form of PTH receptor utilized can be one which encodes a PTH receptor similar to that of the animal species used, or it can encode the PTH receptor homolog of a different species.

The invention now being generally described, the same will be better understood by reference to certain specific examples which are included herein for illustrative purposes only and are not intended to be limiting of the present invention, except where so stated.

### EXAMPLES

### Example 1

Isolation of Total and Poly-A⁺ RNA

Canine tissues were obtained from the Department of Comparative Medicine and Biology Support, Pfizer Central Research, in accordance with institutional policies and immediately frozen in liquid nitrogen. Total cellular RNA was isolated from frozen canine tissues by CsCl centrifugation (Thiede et al., Endocrinology, 135:929-37 (1994)) and quantitated by absorbance at 260 nm. Poly-A⁺ RNA was enriched from canine tissue total RNA using the poly(A) Quik mRNA isolation kit (Stratagene, La Jolla, Calif.) and quantitated by absorbance at 260 nm.

### Example 2

cDNA Library Construction and Screening

A cDNA library in λTriplEx was constructed from canine kidney RNA by the custom services group of Clontech, Inc. (Palo Alto, Calif.). This library was screened by hybridization (50% formamide, 6x SSC, 7.5x Denhardt's Solution, 0.5% SDS; 42° C.; 18 hr) using a ³²P-labeled DNA fragment (Prime-It RmT Kit; Stratagene) corresponding to the complete coding region of human PTH1 (Schipani, 1993) as probe. Filters were washed 3 times (20 minutes per wash) in 6x SSC/0.1% SDS at room temperature and once (20 minutes) in 6x SSC/0.1% SDS at 55° C. Of the 1×10⁶ clones screened in the primary round, 12 of the approximately 1200 positive clones were subjected to an additional two rounds of hybridization and plaque purification. Following conversion of the individual λTriplEx clones to pTriplEx following manufacturer's protocols, plasmid DNAs were prepared using PerfectPrep (5′ [symbol] 3′, Boulder, Colo.), cDNA insert sizes were determined by restriction digestion, and the two clones with the inserts large enough to encode the PTH1 protein coding region were sequenced.

### Example 3

Sequencing

Dideoxy sequencing was performed on canine PTH1 cDNA containing plasmids following insertion of the AT-2 transposon (Primer Island in vitro transposition kit; Perkin-Elmer, Emeryville, Calif.) using transposon specific primers. Gaps and ambiguities in the sequence were addressed by direct sequencing of required regions using gene specific primers. The sequence has been deposited in GenBank with accession number: AF167095.

### Example 4

PCR Subcloning of Canine PTH1 for Protein Expression

A 1868 bp DNA fragment containing the complete coding region of canine PTH1 was generated by PCR using primers complimentary to the 5′ and 3′ untranslated regions (5′ PO₄-TGCCCAGGATCCACMCTGG and 3′ GTCCACGAGTCCMCCCTGG) of the canine PTH1 cDNA using the full length canine PTH1 pTriplEx clone DNA as template. The 5′ phosphorylated PCR product was ligated into the mammalian expression vector pCR3.1-Uni (Invitrogen, Carlsbad, Calif.) as per manufacturer's instructions and subsequently sequenced to confirm cloning junctions and lack of PCR generated changes in the predicted protein sequence.

### Example 5

Expression of Recombinant Canine PTH1 in CHO Cells

CHO-K1 cells were purchased from ATCC (Rockville, Md.) and maintained in Dulbecco's modified Eagle's medium/F12 (DMEM/F12, Life Technologies, Gaithersburg, Md.) supplemented with 10% fetal calf serum (FCS, Gemini Bio-Products, Calabasas, Calif.). CHO cells were plated in 12 and 24-well plates (Becton Dickinson, Franklin Lakes, N.J.) at 1.6×10⁶ cells/plate in DMEM/F12 supplemented with 10% FCS. 24 hours later, each plate of cells was transfected with 10 μg of the canine PTH1-pCR3.1 expression plasmid using Lipofectamine reagent (Life Technologies). Total volume of DNA-lipofectamine mix was 500 μl/well and 250 μl/well in the 12- and 24-well plates, respectively. After 5 hours, an equal volume of media supplemented with 20% FCS was added to each well and after 24 hours, the media was replaced with growth media. The cells were then allowed to grow for an additional 24 hours, at which time the cells in the 24-well plates were used in the PTH binding assay and the cells in the 12-well plates were used to determine receptor activation by measuring cAMP.

### Example 6

Receptor Ligands

¹²⁵I-human PTH 1-34 amide (2000 Ci/mmol) was purchased from Amersham (Arlington Heights, Ill.). Human PTH 1-34 and human PTHrP 1-34 were purchased from Peninsula Laboratories (San Carlos, Calif.). Canine PTH (1-34) and PTHrP (1-34) (Rosol, 1995) were custom synthesized by Princeton BioMolecules (Columbus, Ohio). Human aldosterone secretion inhibitory factor (1-35), used as a similarly sized non-specific peptide, was purchased from Sigma.

### Example 7

Analysis of Binding to Canine PTH1

Canine PTH1 transiently transfected CHO cells in 24-well plates were subjected to whole cell human ¹²⁵I-PTH (1-34) competition binding. Culture media was removed, cell monolayers washed once each with ice-cold Dulbecco's phosphate buffered saline (Life Technologies) and ice-cold assay buffer (AB; 50 mM Tris-HCl, 100 mM NaCl, 2 mM CaCl_{2,} 5 mM KCl, 0.5% heat inactivated FCS (Life Technologies), and 5% heat-inactivated horse serum (Life Technologies), pH 7.7). Subsequent to adding 225 μl of AB to each well, 25 μl of 10x peptide (10⁻⁴- 10^{31 11} M) in AB containing 800 cpms/μl of ¹²⁵I-human PTH (1-34) was added to each of three wells. Plates were incubated for 2 hours on ice, binding mix removed by aspiration, washed 2x with ice-cold assay AB, cells solubilized in 1% SDS and total bound counts determined. Percent bound ¹²⁵I-PTH (1-34) was calculated using the following equation:

$$\frac{\# bound cpms - \# background cpms}{\# maximum bound cpms - \# background cpms} \times 100.$$

### Example 8

Determination of Cyclic AMP (cAMP)

To measure stimulation of cAMP production, cells transiently transfected with canine PTH1 were rinsed once with test medium (serum-free DMEM/F12 containing 0.2 mM 3-isobutyl-1-methylxanthine (IBMX; Calbiochem, La Jolla, Calif.)), then preincubated with 1 ml/well test medium for 10 minutes at 37° C. This medium was removed and cells were incubated with test medium containing the indicated peptides (10⁻⁷ M) for 15 minutes at 37° C. The wells were aspirated and 0.5 ml/well acid ethanol added (0.2 N HCl in 100% ethanol). Following trituration by pipette, the samples were frozen at −20° C. Upon thawing cAMP was measured using a commercially available ELISA kit (Cayman Chemical, Ann Arbor, Mich.).

### Example 9

Northern Blot Analysis

Northern blot analysis was used to determine the transcript size and tissue distribution of canine PTH1 mRNA. Briefly, 5 μg of canine poly-A⁺ RNAs were separated by electrophoresis in a 0.9% agarose-1.89% formaldehyde gel, transferred to Nytran membrane (Schleicher and Schuell, Keene, N.H.) by capillary blotting in 10x SSC, and hybridized as described above to a ³²P-labelled DNA fragment containing the entire canine PTH1 coding region. Following hybridization, the blot was washed 3 times (20 minutes per wash) in 2x SSC/0.1% SDS at room temperature and once (20 minutes) in 0.3x SSC/0.1% SDS at 55° C. Phosphorimage analysis (Cyclone Storage Phosphor System, Packard, Meriden, Conn.) was used to detect PTH1 transcripts.

### Example 10

Immunohistochemical Localization of Canine PTH1

Canine kidneys (PelFreez; Rogers, Ariz.) were sliced into −2 mm sections and fixed in 4% paraformaldehyde in phosphate buffered saline (PBS). Fixed slices were embedded in paraffin, sectioned, deparaffinized with HistoClear (National Diagnostic, Atlanta, Ga.) and rehydrated through graded ethanol to saline. Portions of canine bones were obtained from the Department of Comparative Medicine and Biology Support, Pfizer Central Research according to institutional policies. Bones were fixed for 3 days in 10% normal buffered formalin, decalcified for 7 days in ImmunoCal (Decal, Conger, N.Y.), sectioned, deparaffinized with HistoClear, and rehydrated through graded ethanol to saline.

Polyclonal antibodies to the rat PTH receptor (against peptide CTLDEAERLTEEELH) were obtained from BabCo (Richmond, Calif.) and were used in conjunction with the HistoGold detection kit (Zymed, South San Francisco, Calif.). Following immunohistochemical detection of the anti-PTH receptor antibodies, sections were counterstained for 1 minute in 0.2% eosin Y, dehydrated through graded ethanol, and mounted in Permount (Fisher, Fair Lawn, N.J.).

All publications and patents mentioned in the above specification are herein incorporated by reference. Various modifications and variations of the described invention will be apparent to those skilled in the art without departing from the scope and spirit of the invention. Although the invention has been described in connection with specific preferred embodiments, it should be understood that the invention as claimed is not limited to such specific embodiments.

That which is claimed is:

1. An isolated proteinaceous molecule having canine PTH1 activity, wherein said proteinaceous molecule comprises an amino acid sequence of SEQ ID NO:2, or an amino acid sequence of SEQ ID NO:2 with one or more conservative substitutions therein.

2. An isolated proteinaceous molecule according to claim 1, wherein said amino acid sequence has zero to three conservative substitutions therein.

3. An isolated proteinaceous molecule according to claim 1, wherein said amino acid sequence comprises an amino acid sequence of SEQ ID NO:2.

4. An isolated DNA molecule encoding a proteinaceous molecule according to claim 1.

5. An isolated DNA molecule encoding a proteinaceous molecule according to claim 3.

6. A recombinant expression vector comprising a DNA molecule according to claim 4.

7. A cell transformed by an expression vector according to claim 6.

8. A cell according to claim 7, wherein said cell is that of a prokaryote.

9. A cell according to claim 7, wherein said cell is that of a eukaryote.

10. A cell according to claim 9, wherein said eukaryote is yeast.

11. A cell according to claim 9, wherein said eukaryote is a mammal.

12. A cell according to claim 11, which is a CHO cell.

13. A method of producing a proteinaceous molecule having an activity of canine PTH1, which comprises culturing a cell according to claim 7 for a time sufficient to produce said proteinaceous molecule.

14. A pharmaceutical composition comprising the proteinaceous molecule according to claim 1 and a pharmaceutically acceptable carrier therefor.

15. A pharmaceutical composition comprising the proteinaceous molecule according to claim 3 and a pharmaceutically acceptable carrier therefor.

16. A specific binding partner that selectively binds to the proteinaceous molecule according to claim 1.

17. A method of discovering ligands for the canine PTH1 protein, said method comprising the steps of:
a) combining a test substance with the proteinaceous molecule according to claim 1;
b) measuring specific binding between said test substance and said proteinaceous molecule; and
c) classifying as a ligand said test substance if it binds to said proteinaceous molecule.

18. A method of discovering modulators of canine PTH1 protein activity, said method comprising the steps of:
a) combining a test substance with the proteinaceous molecule according to claim 1;
b) measuring the activity of said proteinaceous molecule in the presence and absence of said test substance and; and
c) classifying said test substance as a modulator of canine PTH1 activity if it modulates the activity of said proteinaceous molecule.
